(19) United States
(12) Patent Application Publication
(10) Pub. No.: US 2017/0204344 A1
(43) Pub. Date: Jul. 20, 2017

(54) Methods and Systems for Separating Particulates from a Liquid Medium

(71) Applicant: MURADEL PTY LTD, Kew East, Victoria (AU)

(72) Inventors: David Milton Lewis, Kew (AU); Andrew Kwong Lee, Norwood (AU); Theo Kalaitzidis, Queenstown (AU); Andreas Isdepsky, Whyalla (AU)

(21) Appl. No.: 15/321,232

(22) PCT Filed: Jun. 24, 2015
(86) PCT No.: PCT/AU2015/000365

§ 371 Date: Dec. 22, 2016

(30) Foreign Application Priority Data
Jun. 24, 2014 (AU) 2014902412

(51) Int. Cl. C10L 1/02; C02F 1/24; C02F 1/38; C12M 1/26; C11C 1/00; C12N 13/00; C12N 1/12; C12M 1/00; C02F 1/463; C02F 1/461

(52) U.S. Cl. CPC C10L 1/02; C02F 1/463; C02F 1/24; C02F 1/385; C02F 1/46109; C11C 1/00; C12N 13/00; C12N 1/12; C12M 47/02; C12M 27/20; C12M 33/22; C02F 2001/007

(57) ABSTRACT

The present disclosure relates to methods and systems for separating particulates from a liquid medium. Certain embodiments provide a method of continuously separating particulates from a liquid medium, the method comprising exposing a liquid medium comprising particulates to electrolysis so as to cause flocculation of the particulates, and continuously separating the flocculated particulates so produced from the liquid medium, thereby continuously separating the particulates from the liquid medium.

### PRIORITY CLAIM

This application claims priority to Australian provisional patent application number 2014902412 filed on 24 Jun. 2014, the content of which is hereby incorporated by reference.

### FIELD

The present disclosure relates to methods and systems for separating particulates from a liquid medium.

### BACKGROUND

Certain industries require the separation of suspended particles from large volumes of media. For example, the harvesting of microalgae and/or cyanobacteria for the production of biofuels typically requires the separation of the microalgae and/or cyanobacteria from open ponds, lakes and reservoirs. Municipal wastewater contains a high concentration of total suspended solids, which need to be removed as part of a water treatment process.

Whilst a variety of methods exist to separate such particles, from a commercial perspective a low operating and energy cost are the most important requirements. These problems are exacerbated when the suspended particles need to be removed from large amounts of liquid media, such as large volumes of water.

In many cases, one of the most efficient methods for removing suspended particles from liquid media is coagulation or flocculation. Flocculation can be induced by the addition of flocculants, including inorganic salts of Al or Fe, polymers such as polyacrylamide, or through processes such as auto-flocculation, bio-flocculation, and ultrasound procedures.

However, methods for the use of flocculation to remove suspended particles typically have relatively high operating costs, particularly when this method is used to remove suspended particles from large volumes of liquid media. In addition, the separation of flocculated particles from large amounts of liquid media also adds significantly to the costs of the process.

Accordingly, there is a need for improved methods for the removal of suspended particles from liquid media using flocculation. The present disclosure relates to a system and process for separation of suspended particles using electroflocculation.

### SUMMARY

The present disclosure relates to methods and systems for separating particulates from a liquid medium.

Certain embodiments of the present disclosure provide a method of continuously separating particulates from a liquid medium, the method comprising:
(i) exposing a liquid medium comprising particulates to electrolysis so as to cause flocculation of the particulates; and
(ii) continuously separating the flocculated particulates so produced from the liquid medium;
thereby continuously separating the particulates from the liquid medium.

Certain embodiments of the present disclosure provide a concentrated microalgal and/or cyanobacterial biomass separated according to a method as described herein.

Certain embodiments of the present disclosure provide a method of concentrating a microalgal and/or cyanobacterial biomass in a liquid medium, the method comprising:
(i) exposing a liquid medium comprising suspended microalgae and/or cyanobacteria to electrolysis so as to cause flocculation of the microalgae and/or cyanobacteria; and
(ii) continuously separating the flocculated microalgae and/or cyanobacteria so produced from the liquid medium;
thereby concentrating the microalgal and/or cyanobacterial biomass.

Certain embodiments of the present disclosure provide a method of continuously removing particulates from waste water so as to treat the water, the method comprising:
(i) exposing waste water comprising particulates to electrolysis so as to cause flocculation of the particulates; and
(ii) continuously separating the flocculated particulates so produced from the waste water so as to remove the particulates;
thereby treating the waste water.

Certain embodiments of the present disclosure provide a method of converting a microalgal and/or cyanobacterial biomass to a liquid fuel and/or a precursor of a liquid fuel, the method comprising:
(i) producing a microalgal and/or cyanobacterial biomass by separating suspended microalgal and/or cyanobacterial particulates from a liquid medium by a method as described herein; and
(ii) converting the microalgal and/or cyanobacterial biomass to a liquid fuel and/or a precursor of a liquid fuel.

Certain embodiments of the present disclosure provide a method of converting a microalgal and/or cyanobacterial biomass to a liquid fuel and/or a precursor of a liquid fuel, the method comprising:
(i) producing a microalgal and/or cyanobacterial biomass by separating suspended microalgae and/or cyanobacteria from a liquid medium by exposing the liquid medium comprising suspended microalgae and/or cyanobacteria to electrolysis so as to cause flocculation of the microalgae and/or cyanobacteria; and
(ii) continuously separating the flocculated microalgae and/or cyanobacteria from the liquid medium to produce a microalgal and/or cyanobacterial biomass; and
converting the microalgal and/or cyanobacterial biomass to a liquid fuel and/or a precursor of a liquid fuel.

Certain embodiments of the present disclosure provide a liquid fuel, and/or a precursor of a liquid fuel produced by a method of converting a microalgal and/or cyanobacterial biomass to a liquid fuel and/or a precursor of a liquid fuel as described herein.

Certain embodiments of the present disclosure provide a system for continuously separating particulates from a liquid medium, the system comprising:
(i) one or more electroflocculating cells for receiving liquid medium comprising particulates to produce electroflocculated particulates;
(ii) one or more mixing units to mix the liquid medium comprising the electroflocculated particulates; and
(iii) one or more separators for continuously separating the electroflocculated particulates from the mixed liquid medium.

Certain embodiments of the present disclosure provide a method of continuously separating particulates from a liquid medium, the method comprising producing electroflocculated particulates from particulates in a liquid medium and continuously separating the electroflocculated particulates so produced from the liquid medium, thereby continuously separating the particulates from the liquid medium.

Certain embodiments of the present disclosure provide a combination product comprising the following components:
one or more electroflocculating cells for receiving liquid medium comprising particulates to produce electroflocculated particulates;
one or more mixing units to mix the liquid medium comprising the electroflocculated particulates; and
one or more separators for continuously separating the electroflocculated particulates from the mixed liquid medium;
wherein the components are provided separately, or as two or more integrated components, for separating particulates from a liquid medium.

Certain embodiments of the present disclosure provide a method of separating particulates from a liquid medium using a system as described herein.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows a schematic top and side view of the overall layout of a harvesting unit for microalgae according to one embodiment.

FIG. 2 shows a schematic side view (A) and a top view (B) of the electro flocculating unit according to one embodiment.

FIG. 3 shows a schematic view of a floating ramp for separating electroflocculated particulates according to one embodiment.

### DETAILED DESCRIPTION

The present disclosure relates to methods and systems for separating particulates from a liquid medium.

Certain disclosed embodiments provide methods and systems that have one or more advantages. For example, some of the advantages of certain embodiments disclosed herein include one or more of the following: a system or process that allows continuous separation of flocculated suspended particles; a system or process that provides a reduced running cost and/or a reduced operating cost; a system or process that provides a low operating and/or energy cost due to one or more of the use of floc-flotation and settling, static mixing, reduction of potential electrode scaling and a continuous mode of operation; a system or process that is suitable for separation of suspended particles from large volumes of media; a system or process that is suitable for continuous recycling of media; a system or process that provides increased concentrations of suspended particles to be obtained; a process for the separation of suspended particles that does not introduce unnecessary anions which can result in the lowering of pH of the medium; a system or process that when used for the separation of microalgae can be used to achieve a concentration that is suitable for the subsequent steps in its processing to a biofuel (or a precursor thereof); a system that is relatively simple and as such involves modest capital investment; a system or process that permits the separation of organic or inorganic suspended particles; to address one or more problems and/or to provide one or more advantages, or to provide a commercial alternative. Other advantages of certain embodiments are disclosed herein or may be appreciated in practicing one or more embodiments.

Certain embodiments of the present disclosure provide a method of continuously separating particulates from a liquid medium.

The term “continuously separating”, or variants thereof used herein such as “continuous separation”, means a process of separation of particulates from a liquid medium where the flocculation of the particulates and the separation of the flocculated particulates so produced occurs as a substantially continuous process. The term does not preclude the inclusion of additional steps between flocculation and separation, for example, mixing occurring of flocculated particulates prior to separation, nor does the term preclude the fact that the combined process of flocculation and separation may be stopped at any time and subsequently being restarted.

Certain embodiments of the present disclosure provide a method of separating particulates from a liquid medium, the method comprising:
(i) exposing a liquid medium comprising particulates to electrolysis so as to cause flocculation of the particulates; and
(ii) separating the flocculated particulates so produced from the liquid medium;
   wherein the separation of the flocculated particulates from the liquid medium occurs substantially continuously.

Certain embodiments of the present disclosure provide a method of continuously separating particulates from a liquid medium, the method comprising:
(i) exposing a liquid medium comprising particulates to electrolysis so as to cause flocculation of the particulates; and
(ii) continuously separating the flocculated particulates so produced from the liquid medium;
   thereby continuously separating the particulates from the liquid medium.

Certain embodiments of the present disclosure provide a method of continuously separating particulates from a liquid medium, the method comprising:
(i) exposing a continuous stream of liquid medium comprising particulates to electrolysis so as to cause flocculation of the particulates; and
(ii) separating the flocculated particulates so produced from the liquid medium;
   thereby continuously separating the particulates from the liquid medium.

In certain embodiments, a method as described herein further comprises exposing the liquid medium after the particulates have been removed to one or more rounds of further electrolysis and separation. In this way, recycling of the liquid medium can occur. In certain embodiments, the method involves a continuous recycling of the liquid medium.

In certain embodiments, the liquid medium comprises a substantially aqueous medium. For example, the liquid medium may be water comprising microalgae and/or cyanobacteria for harvesting or waste water containing one or more particulates for removal, for example to treat the waste water.

In certain embodiments, the liquid medium is drawn from a pond, a lake, a lagoon, a pool, or a tank. Other sources are contemplated.

In certain embodiments, the particulates comprise microalgae and/or cyanobacteria. Other types of particulates are contemplated including organic and inorganic particulates that have an overall negative charge. Typically, the particulates are of a size greater than 2 microns, and are suspended in the liquid medium. In this regard, while the particulates are suspended in the liquid medium it will be appreciated that such suspended particulates can settle over a period of time.

For example, microalgae may be being propagated for harvesting for the production of a biofuel (or a precursor of a biofuel). Methods for propagating microalgae and/or cyanobacteria, including large scale propagation, are known in the art. For example, methods for large scale propagation of microalgae are as described in Fon Sing S., Isdepsky A. and D. M. Lewis (2014) “Continuous recycle of medium for the mass production of Tetraselmis sp. in open ponds under increasing salinity.” Bioresource Technology 161, 47-54.

In certain embodiments, the method comprises electroflocculation.

In certain embodiments, the electrolysis comprises electrolysis using more one or more metal and/or metal alloy anodes. In certain embodiments, the metal and/or metal alloy anodes comprise one or more of aluminium, steel, lead and zinc. In this way, a sacrificial anode can be utilised to release anions that neutralise or reduce a negative charge associated with the suspended particles.

In certain embodiments, the method comprises continuous electrolysis. In certain embodiments, the method comprises continuous electroflocculation.

In certain embodiments, the method comprises discontinuous electrolysis or pulsed electrolysis.

In certain embodiments, the electrolysis comprises a plurality of electrodes. In certain embodiments, the electrolysis comprises a plurality of cathodes. In certain embodiments, the electrolysis comprises a plurality of anodes. In certain embodiments, the electrolysis comprises a plurality of cathodes and anodes. In certain embodiments, the electrolysis comprises a plurality of pairs of cathodes and anodes. In certain embodiments, the electrolysis comprises electrolysis using 4 or more electrodes.

In certain embodiments, the electrolysis comprises alternating current electrolysis. In certain embodiments, the electrolysis comprises electrolysis using alternating current.

In certain embodiments, the electrolysis comprises direct current electrolysis. In certain embodiments, the electrolysis comprises electrolysis using direct current.

In certain embodiments, the electrolysis comprises a current density of 1 Am⁻² to 1000 Am⁻². Other current densities are contemplated.

In certain embodiments, the electrolysis comprises a current density of 1 Am⁻² to 1000 Am⁻², 10 Am⁻² to 1000 Am⁻², 100 Am⁻² to 1000 Am⁻², 1 Am⁻² to 100 Am⁻², or 1 Am⁻² to 10 Am⁻².

In certain embodiments, the electrolysis comprises a voltage of 1 V to 240 V. Other voltages are contemplated.

In certain embodiments, the method comprises use of one or more electrolysis cells or units. In certain embodiments, the method comprises use of a single electrolysis cell or unit. In certain embodiments, the method comprises use of a plurality of electrolysis cells or units. In this regard, these terms refer to a section/area/region where electrolysis may occur. The units may be arranged in a serially and/or parallel manner.

In certain embodiments, the method further comprises the introduction of dissolved gas or bubbles into the liquid medium. In certain embodiments, the dissolved gas or bubbles are introduced into the liquid medium during and/or after electrolysis. In certain embodiments, the dissolved gas or bubbles are introduced into the liquid medium prior to, and/or during, separation of the flocculated particulates. In certain embodiments, the dissolved gas or bubbles are introduced into the liquid medium at one or more of prior to, during and after separation of the flocculated particulates.

In certain embodiments, the method comprises a flow control device to control the flow of the liquid medium. In certain embodiments, the flow control device comprises a fluid control valve or a fluid control regulator.

In certain embodiments, the method comprises providing a discontinuous stream of the liquid medium.

In certain embodiments, the method comprises providing a continuous stream of the liquid medium.

Certain embodiments of the present disclosure provide a method of continuously separating particulates from a liquid medium, the method comprising:
(i) exposing a continuous stream of liquid medium comprising particulates to electrolysis so as to cause flocculation of the particulates; and
(ii) separating the flocculated particulates so produced from the liquid medium;
   thereby continuously separating the particulates from the liquid medium.

In certain embodiments, a fluid control device is used to provide a continuous stream of the liquid medium to the system.

In certain embodiments, the liquid medium is provided under pressure.

In certain embodiments, the method comprises providing a hydrostatic head of the liquid medium. In certain embodiments, a stream of the liquid medium is provided by a hydrostatic head of the liquid medium.

In certain embodiments, the method comprises mixing of the flocculated particulates prior to separation of flocculated particulates. In certain embodiments, the mixing comprises static mixing of the flocculated particulates. Other forms of mixing the flocculated particles in the liquid medium are contemplated. The use of static mixing improves the efficiency of floc-formation.

In certain embodiments, the static mixing comprises up and down static mixing. In certain embodiments, the static mixing comprises side to side static mixing. In certain embodiments, the static mixing comprises up and down static mixing and side to side static mixing.

In certain embodiments, the mixing of the flocculated particulates comprises use of one or more baffles.

In certain embodiments, the separating of the flocculated particulates comprises separating of the flocculated particulates into floating flocculated particulates. In certain embodiments, the separating of the flocculated particulates comprises separating of the flocculated particulates into settled or settling flocculated particulates.

In certain embodiments, the separating of the flocculated particulates comprises separating of the flocculated particulates into floating flocculated particulates and settled or settling flocculated particulates. The collection of suspended particles from both streams may provide improvements in overall harvesting efficiency.

In certain embodiments, the method comprises directing the liquid medium into floating flocculated particulates and settled/settling flocculated particulates. In certain embodiments, the method comprises directing the liquid medium into two streams of floating flocculated particulates and settled/settling flocculated particulates.

In certain embodiments, the separating comprises a separation device to separate floating flocculated particulates. In certain embodiments, the separation device comprises a ramp.

In certain embodiments, the separating comprises a floating separation device to separate floating flocculated particulates. In certain embodiments, the floating separation device comprises a floating ramp. Other types of floating separation devices are contemplated. In certain embodiments, the buoyancy of the floating separation device is adjustable. In certain embodiments, the floating separation device comprises an adjustable air chamber to provide a desired amount of flotation. In certain embodiments, the method comprises use of a flotation device to direct the liquid medium into floating flocculated particulates and settled/settling flocculated particulates. In certain embodiments, the buoyancy of the floating separation device is automatically adjustable.

In certain embodiments, the floating separation device is partially attached or connected to one or more sidewalls of a channel to permit up/down movement according to the water level. In certain embodiments, this permits maintaining a fixed angle with the surface of the liquid medium.

In certain embodiments, the separating of flocculated particulates comprises centrifuging of the floating flocculated particulates. Methods for centrifugation are known in the art.

In certain embodiments, the method comprises use of a settler to separate settled or settling flocculated particulates. In certain embodiments, the method comprises use of a lamella settler to further separate/concentrate the settled or settling flocculated particulates.

In certain embodiments, the method comprises mixing flocculated particles and continuously separating floating flocculated particulates from the liquid medium and/or continuously separating settled or settling flocculated particulates from the liquid medium

In certain embodiments, the method is used to produce a biomass of separated microalgae and/or cyanobacteria. In certain embodiments, the biomass so produced has a dry weight concentration of 5 kgm⁻³ or more. In certain embodiments, the biomass so produced has a dry weight concentration of 10 kgm⁻³ or more. In certain embodiments, the biomass so produced has a dry weight concentration of 15 kgm⁻³ or more. In certain embodiments, the biomass so produced has a dry weight concentration of 20 kgm⁻³ or more. In certain embodiments, the biomass so produced has a dry weight concentration of 25 kgm⁻³ or more, or 30 kgm⁻³ or more.

In certain embodiments, the method is used separate microalgae and/or cyanobacteria from an aqueous medium, to concentrate a biomass, to remove particulates from waste water, and to produce a biomass of a desired product. Other uses are contemplated.

Certain embodiments of the present disclosure provide a method of continuously separating particulates from a liquid medium, the method comprising:
exposing a liquid medium comprising particulates to electrolysis so as to cause flocculation of the particulates;
mixing the flocculated particles;
continuously separating floating flocculated particulates so produced from the liquid medium; and/or
continuously separating settled or settling flocculated particulates so produced from the liquid medium
   thereby continuously separating the particulates from the liquid medium.

Certain embodiments of the present disclosure provide a method of continuously separating particulates from a liquid medium, the method comprising:
exposing a liquid medium comprising particulates to electrolysis so as to cause flocculation of the particulates;
mixing the flocculated particles; and
continuously separating floating flocculated particulates and/or settled or settling flocculated particulates from the liquid medium; thereby continuously separating the particulates from the liquid medium.

Certain embodiments of the present disclosure provide a method of continuously separating particulates from a liquid medium, the method comprising producing electroflocculated particulates from particulates in a liquid medium and continuously separating the electroflocculated particulates so produced from the liquid medium, thereby continuously separating the particulates from the liquid medium.

Certain embodiments of the present disclosure provide particulates separated by a method as described herein.

Certain embodiments of the present disclosure provide a method of concentrating particulates in a liquid medium, the method comprising:
(i) exposing a liquid medium comprising particulates to electrolysis so as to cause flocculation of the particulates; and
(ii) continuously separating the flocculated particulates so produced from the liquid medium;
thereby concentrating the particulates.

Certain embodiments of the present disclosure provide a biomass of microalgae and/or cyanobacteria separated according to a method as described herein.

Certain embodiments of the present disclosure provide a method of concentrating a microalgal and/or cyanobacterial biomass in a liquid medium, the method comprising:
(i) exposing a liquid medium comprising suspended microalgae and/or cyanobacteria to electrolysis so as to cause flocculation of the microalgae and/or cyanobacteria; and
(ii) continuously separating the flocculated microalgae and/or cyanobacteria so produced from the liquid medium;
thereby concentrating the microalgal and/or cyanobacterial biomass.

In certain embodiments, the method as described herein is used to remove particulates from waste water.

Certain embodiments of the present disclosure provide a method of continuously removing particulates from waste water so as to treat the water, the method comprising:
(i) exposing waste water comprising particulates to electrolysis so as to cause flocculation of the particulates; and
(ii) continuously separating the flocculated particulates so produced from the stream of waste water so as to remove the particulates;
thereby treating the waste water.

Certain embodiments of the present disclosure provide a method of converting a microalgal and/or cyanobacterial biomass to a liquid fuel and/or a precursor of a liquid fuel, by separating the microalgal and/or cyanobacterial biomass as described herein and converting the microalgal and/or cyanobacterial biomass to a liquid fuel and/or a precursor of a liquid fuel.

Certain embodiments of the present disclosure provide a method of converting a microalgal and/or cyanobacterial biomass to a liquid fuel and/or a precursor of a liquid fuel, the method comprising:
(i) producing a microalgal and/or cyanobacterial biomass by separating microalgal and/or cyanobacterial particulates from a liquid medium according to a method as described herein; and
(ii) converting the microalgal and/or cyanobacterial biomass to a liquid fuel and/or a precursor of a liquid fuel.

Methods for converting a microalgal and/or cyanobacterial biomass to a liquid fuel and/or a precursor of a liquid fuel are known in the art, for example as described in Barreiro, D. L.; Prins, W.; Ronsse, F.; Brilman, W. (2013), “Hydrothermal liquefaction (HTL) of microalgae for biofuel production: State of the art review and future prospects” Biomass and Bioenergy, vol. 53, pp. 113-127.

Certain embodiments of the present disclosure provide a method of converting a microalgal and/or cyanobacterial biomass to a liquid fuel and/or a precursor of a liquid fuel, the method comprising:
(i) producing a microalgal and/or cyanobacterial biomass by continuously separating suspended microalgae and/or cyanobacteria from a liquid medium by exposing the liquid medium comprising suspended microalgae and/or cyanobacteria to electrolysis so as to cause flocculation of the microalgae and/or cyanobacteria; and
(ii) separating the flocculated microalgae and/or cyanobacteria from the liquid medium to produce a microalgal and/or cyanobacterial biomass; and
converting the microalgal and/or cyanobacterial biomass to a liquid fuel and/or a precursor of a liquid fuel.

Methods for converting a microalgal and/or cyanobacterial biomass to a liquid fuel and/or a precursor of a liquid fuel are known in the art.

In certain embodiments, the converting of the microalgal and/or cyanobacterial biomass to a liquid fuel and/or a precursor of a liquid fuel comprise one of more of cell disruption of the microalgae and/or cyanobacteria, solvent extraction, catalytic conversion and refining.

In certain embodiments, the converting of the microalgal and/or cyanobacterial biomass to a liquid fuel and/or a precursor of a liquid fuel comprises hydrothermal liquefaction or thermochemical liquefaction. For example, hydrothermal liquefaction is a biomass to liquid conversion process carried out at near sub- or super-critical temperature (280° C. to 370° C., 10 MPa to 25 MPa).

Certain embodiments of the present disclosure provide liquid fuel and/or a precursor of a liquid fuel, produced by a method as described herein.

Certain embodiments of the present disclosure provide a system/assembly for continuously separating particulates from a liquid medium.

Certain embodiments of the present disclosure provide a system/assembly for continuously separating particulates from a liquid medium using a method as described herein.

Certain embodiments of the present disclosure provide a system for continuously separating particulates from a liquid medium, the system comprising:
(i) one or more electroflocculating cells for receiving liquid medium comprising particulates to produce electroflocculated particulates;
(ii) one or more mixing units to mix the liquid medium comprising the electroflocculated particulates; and
(iii) one or more separators for continuously separating the electroflocculated particulates from the mixed liquid medium.

In certain embodiments, the system further comprises a means for returning the liquid medium after separation of the electroflocculated particulates for one or more rounds of further electrolysis, mixing and/or separation. In this way, recycling of the liquid medium can occur. For example, the liquid medium may be returned to a point in the system prior to any one or more of the electroflocculating cells, the mixing unit or the separators. In certain embodiments, the liquid medium may be returned to the original source holding the liquid medium, such as a pond, a lake, a lagoon, a pool, or a tank.

In certain embodiments, the liquid medium comprises a substantially aqueous medium. For example, the liquid medium may be water comprising microalgae and/or cyanobacteria for harvesting or waste water containing one or more particulates for removal, for example to treat the waste water.

In certain embodiments, the liquid medium is drawn from a pond, a lake, a lagoon, a pool, or a tank. Other sources are contemplated. In certain embodiments, system comprises a means for drawing the liquid medium from a pond, a lake, a lagoon, a pool, or a tank.

In certain embodiments, the system comprises a means for providing the liquid medium under pressure.

In certain embodiments, the system comprises a means for providing a hydrostatic head of the liquid medium. In certain embodiments, a source of the liquid medium provides a hydrostatic head to assist flow of the liquid medium through the system.

In certain embodiments, the particulates comprise microalgae and/or cyanobacteria. Other types of particulates are contemplated.

For example, microalgae and/or cyanobacteria may be propagated for harvesting for the production of a biofuel (or a precursor of a biofuel). Methods for propagating microalgae and/or cyanobacteria, including large scale propagation, are known in the art. For example, methods for large scale propagation of microalgae and/or cyanobacteria are as described in, for example, Spirulina Platensis Arthrospira: Physiology, Cell-Biology And Biotechnology (2002) edited by Avigad Vonshak CRC Press, 12 Apr. 2002.

In certain embodiments, the electroflocculating cells comprise more one or more metal and/or metal alloy anodes. In certain embodiments, the metal and/or metal alloy anodes comprise one or more of aluminium, steel, lead and zinc.

In certain embodiments, the electroflocculating cells comprise a plurality of electrodes. In certain embodiments, the electroflocculating cells comprise a plurality of cathodes. In certain embodiments, the electroflocculating cells comprise a plurality of anodes. In certain embodiments, the electroflocculating cells comprise a plurality of cathodes and anodes. In certain embodiments, the electroflocculating cells comprise a plurality of pairs of cathodes and anodes. In certain embodiments, the electroflocculating cells comprise 4 or more electrodes.

In certain embodiments, the system uses electrolysis using more one or more metal and/or metal alloy anodes. In certain embodiments, the metal and/or metal alloy anodes comprise one or more of aluminium, steel, lead and zinc. In this way, a sacrificial anode can be utilised to release anions that neutralise or reduce a negative charge associated with the suspended particles.

In certain embodiments, the electroflocculating cells use alternating current electrolysis.

In certain embodiments, the electroflocculating cells use direct current electrolysis.

In certain embodiments, the system uses alternating current electrolysis. In certain embodiments, the system uses direct current electrolysis.

In certain embodiments, the electroflocculating cells use a current density of 1 Am⁻² to 1000 Am⁻². Other current densities are contemplated. In certain embodiments, the system uses electrolysis comprising a current density of 1 Am⁻² to 1000 Am⁻².

In certain embodiments, the electrolysis comprises a current density of 1 Am⁻² to 1000 Am⁻², 10 Am⁻² to 1000 Am⁻², 100 Am⁻² to 1000 Am⁻², 1 Am⁻² to 100 Am⁻², or 1 Am⁻² to 10 Am⁻².

In certain embodiments, the electroflocculating cells use a voltage of 1 V to 240 V. Other voltages are contemplated. In certain embodiments, the system uses electrolysis comprising a voltage of 1 V to 240 V.

In certain embodiments, the system comprises one or more electrolysis cells or units. In certain embodiments, the system comprises a single electrolysis cell or unit. In certain embodiments, the system comprises a plurality of electrolysis cells or units. The cells or units may be arranged in a serial and/or parallel manner.

In certain embodiments, the system comprises use of a plurality of electrolysis cells or units.

In certain embodiments, the system comprises a means for introducing dissolved gas or bubbles into the liquid medium. Sources for introducing gas or bubbles are known in the art.

In certain embodiments, the dissolved gas or bubbles are introduced into the liquid medium before the electroflocculating units. In certain embodiments, the dissolved gas or bubbles are introduced into the liquid medium after the electroflocculating units. In certain embodiments, the dissolved gas or bubbles are introduced into the liquid medium before the mixing units. In certain embodiments, the dissolved gas or bubbles are introduced into the liquid medium after the mixing units. In certain embodiments, the dissolved gas or bubbles are introduced into the liquid medium before the separators.

In certain embodiments, the system comprises a flow control device to control the flow of the liquid medium. In certain embodiments, the flow control device comprises a control valve.

In certain embodiments, the one or more electroflocculating cells and the one or mixing units are co-located. For example, the system may comprise a tank or chamber holding the one or more electroflocculating cells and the one or mixing units.

In certain embodiments, the one or more electroflocculating cells and the one or mixing units are separate. In this embodiment, for example a pipe connecting the electroflocculating cells to the mixers may be used.

In certain embodiments, the system uses mixing of the flocculated particulates prior to separation of flocculated particulates. In certain embodiments, the system uses static mixing of the flocculated particulates. Other forms of mixing the flocculated particles in the liquid medium are contemplated. The use of static mixing improves the efficiency of floc formation.

In certain embodiments, the one or more mixing units comprise static mixers. In certain embodiments, the static mixers comprise up and down baffles. In certain embodiments, the static mixing comprises side to side static mixing. In certain embodiments, the static mixers comprises up and down static mixers and side-to-side static mixers. In certain embodiments, the one or more mixing units comprise one or more baffles.

In certain embodiments, the system comprises a means for providing a discontinuous stream of the liquid medium.

In certain embodiments, the system comprises a means for providing a continuous stream of the liquid medium. For example, a fluid control device may be used to provide a continuous stream of the liquid medium to the system.

In certain embodiments, the system comprises use of a hydrostatic head of the liquid medium. In certain embodiments, a stream of the liquid medium is provided by a hydrostatic head of the liquid medium.

In certain embodiments, the one or more separators comprise a separation device to separate floating electroflocculated particulates. In certain embodiments, the one or more separators comprise a separation device to separate floating electroflocculated particulates and settled or settling electroflocculated particulates.

In certain embodiments, the one or more separators comprise a settler to separate settled electroflocculated particulates. In certain embodiments, the settler comprises a lamella settler.

In certain embodiments, the system separates the flocculated particulates into floating flocculated particulates. In certain embodiments, the system separates the flocculated particulates into settled or settling flocculated particulates. In certain embodiments, the system separates the flocculated particulates into floating flocculated particulates and settled or settling flocculated particulates. The collection of suspended particles from both streams may provide improvements in overall harvesting efficiency.

In certain embodiments, the system method directs the liquid medium into floating flocculated particulates and settled/settling flocculated particulates. In certain embodiments, the system directs the liquid medium into two streams of floating flocculated particulates and settled/settling flocculated particulates.

In certain embodiments, the one or more separators comprise one or more floating separation devices. In certain embodiments, the floating separation device comprises a floating ramp. Other types of floating separation devices are contemplated. In certain embodiments, the buoyancy of the floating separation device is adjustable. In certain embodiments, the floating separation device comprises an adjustable air chamber to provide a desired amount of flotation. In certain embodiments, the system uses a flotation device to direct the liquid medium into floating flocculated particulates and settled/settling flocculated particulates.

In certain embodiments, the floating separation device is partially attached or connected to one or more side walls of a channel to permit up/down movement according to the water level. In certain embodiments, this permits maintaining a fixed angle with the surface of the liquid medium.

In certain embodiments, the system further comprises one or more centrifuges to further separate or concentrate the particulates. In certain embodiments, the system comprises centrifuging of the floating flocculated particulates after separation.

In certain embodiments, the system comprises a settler. In certain embodiments, the system comprises a lamella settler. In certain embodiments, the system comprises use of a lamella settler to further separate/concentrate settled or settling flocculated particulates.

In certain embodiments, the system further comprises a centrifuge for settled or settling flocculated particulates.

Certain embodiments of the present disclosure provide a system for continuously separating particulates from a liquid medium, the system comprising:
one or more electroflocculating cells for receiving liquid medium comprising particulates to produce electroflocculated particulates;
one or more mixing units to mix the liquid medium comprising the electroflocculated particulates; and
either or both of a floating separator and a settler.

Certain embodiments of the present disclosure provide a system for continuously separating particulates from a liquid medium, the system comprising:
one or more electroflocculating cells for receiving liquid medium comprising particulates to produce electroflocculated particulates;
one or more mixing units to mix the liquid medium comprising the electroflocculated particulates; and
either or both of a floating separator for continuously separating floating electroflocculated particulates from the mixed liquid medium and a settler for continuously separating settled and/or settling electroflocculated particulates from the liquid medium.

In certain embodiments, the system is used to produce a biomass of separated microalgae and/or cyanobacteria. In certain embodiments, the biomass so produced has a dry concentration of 5 kgm⁻³ or more. In certain embodiments, the biomass so produced has a dry concentration of 10 kgm⁻³ or more. In certain embodiments, the biomass so produced has a dry concentration of 15 kgm⁻³ or more. In certain embodiments, the biomass so produced has a dry concentration of 20 kgm⁻³ or more. In certain embodiments, the biomass has a dry concentration of 25 kgm⁻³ or more, or 30 kgm⁻³ or more.

Certain embodiments of the present disclosure provide a method of separating particulates from a liquid medium, the method using a system as described herein.

In certain embodiments, the system is used to separate microalgae and/or cyanobacteria from an aqueous medium, to concentrate a biomass, to remove particulates from waste water, and to produce a biomass of a desired product.

Certain embodiments of the present disclosure provide particulates separated using a system as described herein. Certain embodiments of the present disclosure provide particulates concentrated using a system as described herein.

Certain embodiments of the present disclosure provide a biomass of microalgae and/or cyanobacteria separated using a system as described herein.

In certain embodiments, the system as described herein is used to remove particulates from waste water.

Certain embodiments of the present disclosure provide a combination product comprising one or more of a system as described herein.

Certain embodiments of the present disclosure provide a combination product comprising the following components:
one or more electroflocculating cells for receiving liquid medium comprising particulates to produce electroflocculated particulates;
one or more mixing units to mix the liquid medium comprising the electroflocculated particulates; and
one or more separators for continuously separating the electroflocculated particulates from the mixed liquid medium;
   wherein the components are provided separately, or as two or more combined components, for separating particulates from a liquid medium.

Certain embodiments of the present disclosure provide a method of converting a microalgal and/or cyanobacterial biomass to a liquid fuel and/or a precursor of a liquid fuel, by separating the microalgal and/or cyanobacterial biomass using a system as described herein and converting the microalgal and/or cyanobacterial biomass to a liquid fuel and/or a precursor of a liquid fuel.

Methods for converting a microalgal and/or cyanobacterial biomass to a liquid fuel and/or a precursor of a liquid fuel are known in the art.

In certain embodiments, the converting of the microalgal biomass to a liquid fuel and/or a precursor of a liquid fuel comprises hydrothermal liquefaction or thermochemical liquefaction.

Certain embodiments of the present disclosure provide liquid fuel and/or a precursor of a liquid fuel, produced by a system as described herein.

The present disclosure is further described by the following examples. It is to be understood that the following description is for the purpose of describing particular embodiments only and is not intended to be limiting with respect to the above description.

### Example 1—System and Process for Continuous Separation of Particulates

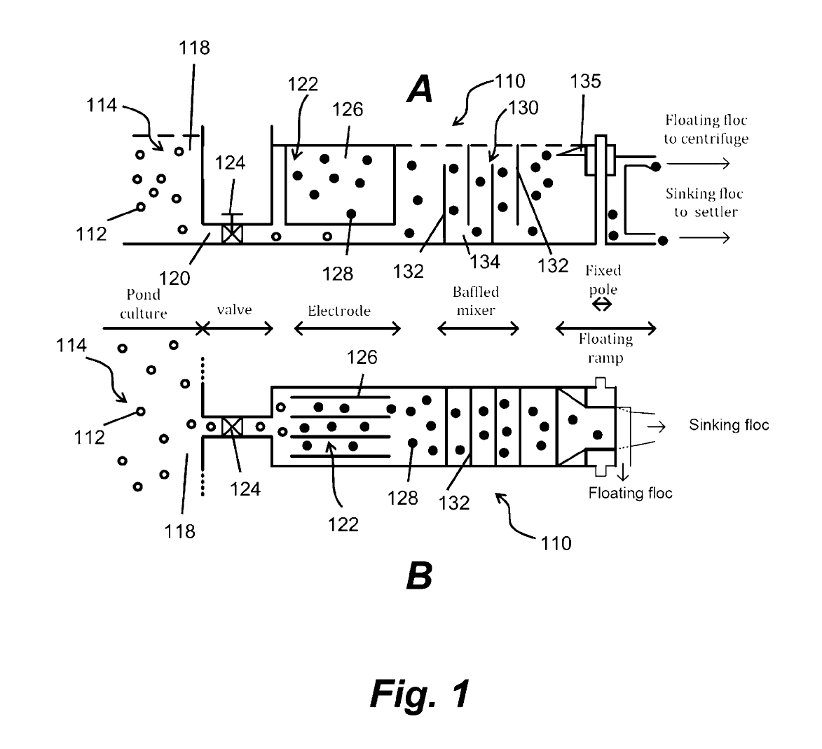

Referring to FIG. 1, there is shown a schematic representation of a side view (FIG. 1A) and a top view (FIG. 1B) of a system or assembly 110 for continuously separating particulates 112 from a liquid medium 114 according to one embodiment. The system 110 uses a continuous flocculation/separation system involving electrolysis.

In the embodiment shown the system 110 is used for continuously separating particulate microalgae or cyanobacteria 112 from aqueous medium 114. Methods for propagating micro-organisms such as microalgae are known in the art. Other types of suspended particles, including organic or inorganic particulates, can be separated using the system and process described herein.

Microalgae and/or cyanobacteria are grown, for example, for use in the production of biomasses as a source of fuel using techniques such as hydrothermal liquefaction or other products such as oleochemicals using cell disruption and solvent extraction. Typically, microalgae and/or cyanobacteria may be grown in a medium 114 to a concentration of about 0.2 to 1.2 kg m⁻³. The system and process described herein provides the ability to obtain increased biomass concentrations.

In the embodiment shown, the microalgae and/or cyanobacteria 112 are continuously separated by exposing a continuous stream of the medium 114 to electrolysis so as to cause flocculation of the particulates 112 to form flocculated particulates 128. The continuously flocculated particulates 128 are separated from the aqueous medium 114, thereby continuously separating the particulate microalgae and/or cyanobacteria 112 from the aqueous medium 114.

The system 110 is located adjacent to a pond or holding unit 118. The system 110 can use a naturally occurring hydrostatic head to provide the media flow through the system. Example of other sources of water with particulates are a lake, a lagoon, a pool, or a tank.

A pipe 120 delivers a stream of the aqueous medium 114 to an electrolysis cell, unit or module 122 with the flow of the medium controlled by a control valve 124 or a similar flow control device. A steady flow rate, using a controlled volume of media, maintains a predetermined water level and residence time for electrolysis and flocculation. In the embodiment shown, the source of water 118 provides a natural hydrostatic head to assist with movement of the water through the system.

The electrolysis unit 122 includes a series of electrodes in the form of metal plates 126 of opposite/alternating electrical polarities. It will be appreciated that other forms of the electrodes 126 may be used. Typically, the electrodes 126, such as the anodes, are made of metals or metal alloys of relatively low toxicity, such as aluminium, iron, stainless steel, lead or zinc. The electrodes 126 may also be made of non-metals, such as graphite. The surface(s) of the electrodes 126 may be bare, oxidised or coated with a different metal or alloy. The shape of the electrodes 126 may be flat plates, as shown in the embodiment in FIG. 1, or be co-centric cylinders, to allow a uniform electric field between electrodes of opposite polarities. Other shapes are contemplated. Typically, the number of electrodes 126 varies from 4 to 100 with the electrodes arranged in mono- or bi-polar mode with or without surface scrapers to remove electrode surface build-ups.

Either alternating current (AC) or direct current (DC) may be supplied to the electrolysis unit 122 as the electrical energy supply for the dissolution of sacrificial metals to provide the ions necessary for flocculation. The alternating current (AC) or the direct current (DC) may be used at a current density ranging from 1 A m⁻² to 1000 A m⁻², and a voltage ranging from 1 V to 240 V. In the case of AC, the waveform can be sinusoidal, rectangular, triangular or any form that can be suitably described by a mathematical formula.

Electroflocculation of the microalgae and/or cyanobacteria 112 is achieved by passing the electrical current between a sacrificial anode (typically aluminium or iron) and the cathode. Hydrogen gas is generated at the cathode and the sacrificial anode releases cations which destabilize microalgae by reducing or neutralizing the negative surface charge, thereby causing flocculation of the microalgae and/or cyanobacteria to produce flocculated particles (also referred to as “flocs”) 128. Typically the flocs 128 settle to the bottom of the vessel or attach to the gas bubbles from the electrodes and float to the surface. Dissolved gas or bubbles may also be introduced during the flocculation process to increase the portion of flocs 128 collected via flotation. Conductivity of the culture media can be adjusted by the addition of acids, alkalis or salts.

In addition to the electrolysis/electroflocculating unit 122, the system 110 further comprises a mixing unit, in the form of a static mixer 130, downstream of the electrolysis/electroflocculating unit 122, to mix the flocculated particulates prior to their separation. The mixer 130 comprises a series of baffles or a static mixing device 132 to increase the amount of mixing and enhance the floc formation during the flow through the unit. Mechanical mixing using a static device allows a uniform mixture of flocculent 128, and the suspended particles 122 also provide momentum for the collision of the particles to form larger flocs 128 and increase the probability of contact among flocs 128. If a baffled system is adopted, the physical dimensions of the baffles are designed to impart optimum mixing during flocculation by forcing the flow through a number of 180° bends 134. These bends 134 direct the flow in a left-right or up-down manner by different baffle arrangements. The number of baffles and the length of the flow path can be varied according to the volumetric flow rate of the media and the construction material of the baffles 132, which are typically made of concrete, brick and mortar, plastic, metal, wood, reinforced resins or any other suitable material. The static mixing may utilise up and/or down static mixing, side to side static mixing, or a combination thereof.

In the embodiment shown, the system 110 uses a separator 135 in the form of floating separation device 135 to separate floating flocculated particulates. In the embodiments shown, the floating separation device comprises a floating ramp 135. The floating ramp 135 located at or near the end of the mixer 134 and divides the out flow into two streams: one carrying the floating flocs attached by the gas bubbles generated during electrolysis—this stream is directed to a centrifuge (not shown). The other stream carrying settling flocs is directed to a lamella settler to be further concentrated. This concentrated secondary underflow is then also centrifuged if required, while the clarified overflow may be recycled. As such, the system 110 permits separating of the flocculated particulates into floating flocculated particulates and/or settled or settling flocculated particulates.

The liquid medium 114 from which the flocs 128 have been separated may then be recycled, if desired. For example, the liquid medium can be returned to the pond or holding unit 118, or alternatively can be returned to a point in the system 110 before the electrolysis unit 122 and/or before the static mixer 130. In this way, the process of separation may expose the liquid medium after the particulates have been removed to one or more rounds of further electrolysis and/or separation.

The system 110 described in this embodiment provides a continuous flocculation/separation system which utilises electricity to dissolve sacrificial metals for the separation with the subsequent media recycled to be reused if desired. The system described may be used to produce a biomass of separated microalgae and/or cyanobacteria with a dry weight concentration of 5 kgm⁻³ or more, and typically a dry weight concentration of up to 30 kgm⁻³ particulates may be achieved. As such, the system and process described herein also provide an efficient dewatering system. A low operating and energy cost may be achieved by the combined use of one or more of floc flotation and settling, incorporation of static mixing in the plant design, minimal electrode scaling and a continuous mode of operation. It will also be appreciated that the system 110 and process described herein may be used for a variety of other purposes, for example to remove particulates from waste water, and to separate other types of particulates from a liquid medium.

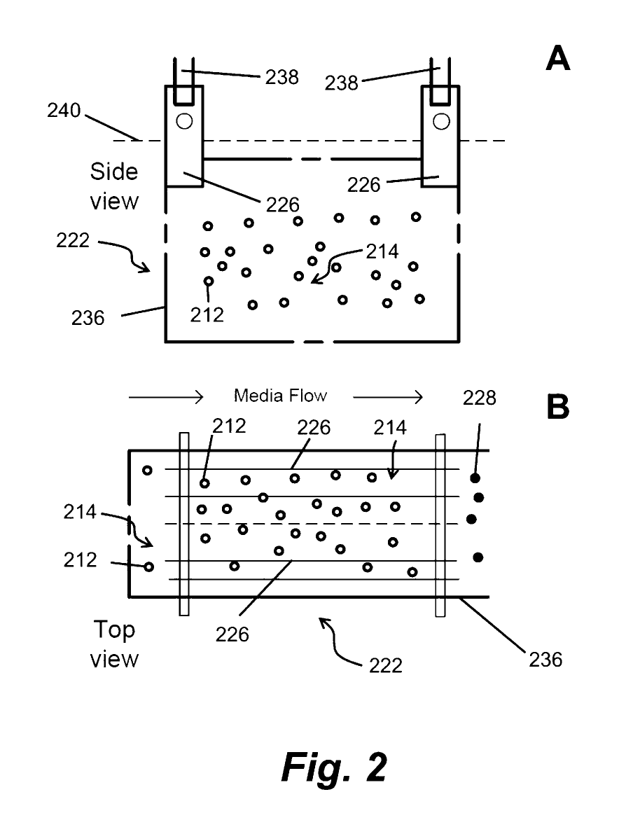

FIG. 2 shows a schematic side and top view of the electrolysis unit 222 according to one embodiment. Referring to FIG. 2A, there is shown a side view of the electrolysis unit 222 in which particulate microalgae 212 are present in an aqueous medium 214 prior to electroflocculation. The electrolysis unit 222 is situated in a flocculation tank 236, which holds the electrolysis unit 222 and also a static mixer (not shown). The flocculation tank 236 can be of any suitable configuration, for example rectangular, cylindrical or spherical in shape, and may be located above or below ground. The construction material of the flocculation tank 236 may from any suitable material, for example reinforced resin, concrete, plastic, or brick and mortar.

The electrolysis unit 222 comprises of a series of electrodes 226 in the form of metal (or metal allow) plates 226 of opposite electrical polarities, connected to an electrical source by connectors 238. The electrodes 226 are immersed below the water line 240 in the aqueous medium 214.

Referring to FIG. 2B, there is shown a top view of the electrolysis unit 222 in which particulate microalgae and/or cyanobacteria 212 are present in an aqueous medium 214. The electrolysis unit 222 is situated in a flocculation tank 236, which holds the electrolysis unit 222 and also a static mixer (not shown). In the embodiment shown, the direction of flow of the liquid medium 214 is parallel to the electrolysis plates 226, although the flow of the liquid medium 214 may also be directed in other directions such as perpendicular or sideways to plates 226. The particulate microalgae and/or cyanobacteria 212 are subsequently subjected to electroflocculation, to form flocculated particulates 228, which are then subject to further mixing. Dissolved gas or bubbles may also be introduced during the flocculation process to increase the portion of flocs 228 collected via flotation. Conductivity of the liquid medium can also be adjusted by the addition of acids, alkalis or salts.

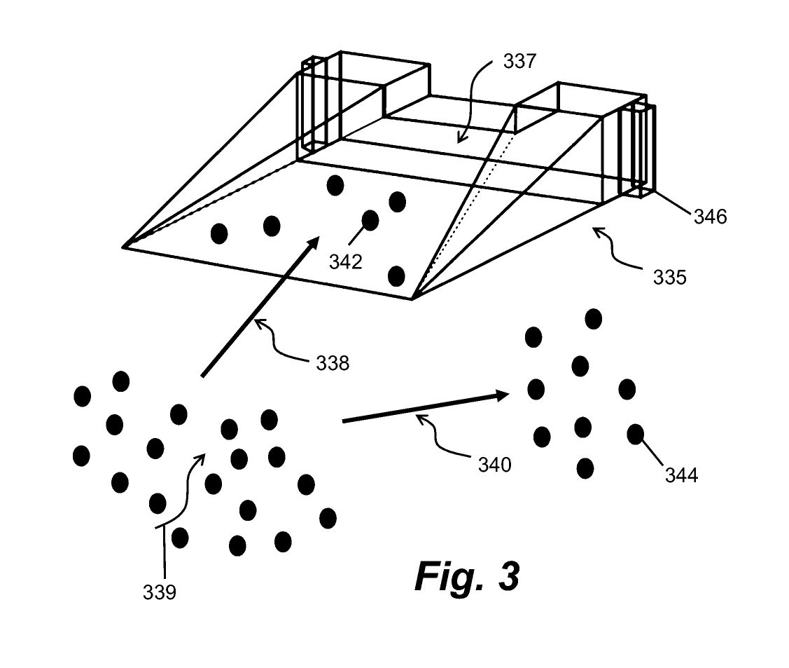

FIG. 3 shows a schematic presentation of a floating ramp 335 according to one embodiment. The floating ramp 335 may be composed of plastic or similar material, which is typically impervious to water. An adjustable air chamber 337 located inside the ramp provides the correct amount of flotation. This ramp 335 is located at the end of the static mixing unit (not shown) to divide the exit flow 339 from the static mixing unit(s) into two streams 338 and 340—the first stream 338 contains floating flocs 342 that may carry gas bubbles formed during electrolysis (and/or actively introduced) and the other stream 340 carries settling flocs 344. The ramp 335 is partially attached to the side walls in the electroflocculation and mixing chamber using protuberances 346 which sliding fit in a channel (not shown) to allow up-down movement according to the water level but still maintaining a fixed angle with the water surface. Processing of the floating flocs 342 and the settling flocs 344 is a described above.

### Example 2—Conversion of Separated Microalgae into a Biofuel

Once flocculated, the concentrated microalgae and/or cyanobacteria separated as described herein is then transferred via pumping for further concentration, e.g. centrifugation, or directly transferred to a conversion process for biofuel production. The conversion process is typically hydrothermal liquefaction, or cell lysis (e.g. hydrodynamic cavitation) for lipid extraction.

Methods for converting microalgae and/or cyanobacteria into biofuels are known in the art, for example as described in Barreiro, D. L.; Prins, W.; Ronsse, F.; Brilman, W. (2013), “Hydrothermal liquefaction (HTL) of microalgae for biofuel production: State of the art review and future prospects” Biomass and Bioenergy, vol. 53, pp. 113-127.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1. A method of continuously separating particulates from a liquid medium, the method comprising:
(i) exposing a liquid medium comprising particulates to electrolysis so as to cause flocculation of the particulates; and
(ii) continuously separating the flocculated particulates so produced from the liquid medium;
thereby continuously separating the particulates from the liquid medium.

2. The method according to claim 1, wherein the method further comprises exposing the liquid medium after the particulates have been separated to one or more rounds of further electrolysis and separation.

3. (canceled)

4. The method according to claim 1, wherein the particulates comprise microalgae and/or cyanobacteria.

5. The method according to claim 1, wherein the electrolysis comprises electrolysis using a sacrificial metal anode.

6-13. (canceled)

14. The method according to claim 1, wherein the method comprises mixing of the flocculated particulates in one or more baffles prior to separation of the flocculated particulates.

15-18. (canceled)

19. The method according to claim 1, wherein the method comprises separating of the flocculated particulates into floating flocculated particulates and settled or settling flocculated particulates.

20. The method according to claim 19, wherein the separating comprises a floating ramp to separate the floating flocculated particulates.

21-22. (canceled)

23. The method according to claim 19, wherein the separating comprises a lamella settler to separate the settled or settling flocculated particulates.

24-27. (canceled)

28. The method according to claim 1, wherein the method is used to produce a biomass of separated microalgae and/or cyanobacteria.

29. The method according to claim 28, wherein the biomass has a dry weight of 5 kgm⁻³ or more.

30-33. (canceled)

34. A method of converting a microalgal and/or cyanobacterial biomass to a liquid fuel and/or a precursor of a liquid fuel, the method comprising:
(i) producing a microalgal and/or cyanobacterial biomass by separating microalgal and/or cyanobacterial particulates from a liquid medium according to claim 1; and
(ii) converting the microalgal and/or cyanobacterial biomass to a liquid fuel and/or a precursor of a liquid fuel.

35-36. (canceled)

37. A liquid fuel and/or a precursor of a liquid fuel produced according to claim 34.

38. A system for continuously separating particulates from a liquid medium, the system comprising:
(i) one or more electroflocculating cells for receiving a liquid medium comprising particulates to produce electroflocculated particulates;
(ii) one or more mixing units to mix the liquid medium comprising the electroflocculated particulates; and
(iii) one or more separators for continuously separating the electroflocculated particulates from the mixed liquid medium.

39. The system according to claim 38, wherein the system further comprises means for returning the liquid medium after separation of the electroflocculated particulates for one or more rounds of further electrolysis, mixing and/or separation.

40. (canceled)

41. The system according to claim 38, wherein the particulates comprise microalgae and/or cyanobacteria.

42. The system according to claim 38, wherein the electroflocculating cells comprises one or more sacrificial metal anodes.

43-53. (canceled)

54. The system according to claim 38, wherein a mixing unit comprises a baffle.

55. (canceled)

56. The system according to claim 38, wherein the one or more separators comprise a separation device to separate floating electroflocculated particulates and a separation device to separate settled or settling electroflocculated particulates.

57. (canceled)

58. The system according to claim 56, wherein the separation device to separate floating electroflocculated particulates comprises a floating ramp.

59. (canceled)

60. The system according to claim 56, wherein the separation device to separate settled or settling electroflocculated particulates comprises a lamella settler.

61-65. (canceled)

66. A method of separating particulates from a liquid medium, the method using a system according to claim 38.

67-71. (canceled)

72. A method of converting a microalgal and/or cyanobacterial biomass to a liquid fuel and/or a precursor of a liquid fuel, the method comprising:
(i) producing a microalgal and/or cyanobacterial biomass by continuously separating suspended microalgae and/or cyanobacteria from a liquid medium by exposing the liquid medium comprising suspended microalgae and/or cyanobacteria to electrolysis so as to cause flocculation of the microalgae and/or cyanobacteria and separating the flocculated microalgae and/or cyanobacteria from the liquid medium to produce a microalgal and/or cyanobacterial biomass; and
(ii) converting the microalgal and/or cyanobacterial biomass to a liquid fuel and/or a precursor of a liquid fuel.
